(19) United States
(12) Patent Application Publication
(10) Pub. No.: US 2024/0325770 A1
(43) Pub. Date: Oct. 3, 2024

(54) METHODS OF TREATING CARTILAGE GRAFTS WITH ALTERNATING ELECTRIC FIELDS

(71) Applicant: NOVOCURE GMBH, Root D4 (CH)

(72) Inventor: LILACH AVIGDOR, Haifa (IL)

(21) Appl. No.: 18/621,869

(22) Filed: Mar. 29, 2024

Related U.S. Application Data
(60) Provisional application No. 63/493,393, filed on Mar. 31, 2023.

(51) Int. Cl. A61N 1/40

(52) U.S. Cl. CPC A61N 1/40

(57) ABSTRACT

Disclosed are methods of treating a subject having a cartilage graft comprising applying alternating electric fields, at a frequency for a period of time, to a target site of the subject, wherein the target site comprises the cartilage graft. Disclosed are methods of increasing N-cadherin expression or promoting chondrogenesis in a subject having a cartilage graft comprising applying alternating electric fields, at a frequency for a period of time, to a target site of the subject. Disclosed are methods of upregulating the Akt/PI3K pathway in a subject having a cartilage graft comprising applying alternating electric fields, at a frequency for a period of time, to a target site of the subject.

### CROSS-REFERENCE TO RELATED APPLICATIONS

This application claims the benefit of U.S. Provisional Patent Application No. 63/493,393, filed Mar. 31, 2023, which is incorporated by reference herein in its entirety.

### BACKGROUND

Cadherins are Ca2+ dependent cell-cell adhesion molecules, whose extracellular domain consists of five extracellular cadherin (EC) repeats. The ECs mediate homophilic interactions between cells expressing identical cadherins and selective heterophilic interactions between cells expressing different cadherins, which induce the cell segregation during tissue morphogenesis and drive cell aggregation.

The extracellular microenvironment transforms from the initial state rich in cell-cell interactions to cell-ECM interactions in the process of tissue development in vivo.

Articular cartilage defects can result from trauma, degeneration or systemic immune diseases, leading to the loss of articular structure and functions. The avascular property of articular cartilage leads to a lack of classic healing cascade, such as coagulation, inflammation, blood invasion, and accumulation of multipotent mesenchymal stem cells (MSCs), which largely limits the self-healing potential of adult articular cartilage.

In the last decades, to provide alternative treatment options to these clinical techniques, enormous efforts have been made to develop cartilage tissue engineering techniques, which show promising application potential.

Examples of treatment options are microfracture methods, autologous chondrocyte implantation (ACI), and peptide therapeutics.

The microfracture method triggers the self-healing capacity of subchondral bone tissue and bears inexpensive, short and simple nature as well as a quick recovery time, which makes it attractive for the repair of small articular cartilage defects (less than 2 cm²).

Autologous chondrocyte implantation (ACI) is the gold-standard treatment for large-size cartilage defects (up to 12 cm²) or when microfracture fails. This technique involves the harvest of chondrocytes from a low-weight-bearing region of the joint by biopsy punch in the first operation, in-vitro amplification of cell population, and transplantation into the debrided cartilage defects in the second operation. This treatment benefits the healing of articular cartilage defects by providing autologous and thus non-immunogenic chondrocytes with minimized complications in donor sites.

N-Cadherin mimetic peptides have been found to direct cell-cell interactions during mesenchymal condensation, which facilitates chondrogenic and osteogenic differentiation of MSCs under the coordinate assistance of other inductive factors and hydrogel structures. A hyaluronic acid hydrogel tethered with the HAVDIGGGC motif can upregulate the chondrogenic genes of encapsulated human MSCs at the early stage of differentiation and promote cartilage matrix deposition compared with the unmodified hydrogel, scrambled peptide-conjugated hydrogel or the hydrogel treated with N-cadherin-specific antibodies.

Peptides can be chemically synthesized, thus bearing higher yielding, lower cost and immunogenicity, which present an attractive group of bioactive agents to chondrogenically functionalize biomaterials for cartilage tissue engineering. Through mimicking the actions of chondrogenesis-related ligands, cell-cell junction molecules and ECM compositions, a large variety of peptides have been designed to trigger desired cellular signaling pathways.

N-cadherins have also been found to play an essential role for mesenchymal cell condensation during chondrogenesis (the process in which cartilage is formed from condensed mesenchyme tissue, which differentiates into chondrocytes and begins secreting the molecules that form the extracellular matrix) through mediating the aggregation of progenitor cells and promoting cell-to-cell interactions.

For example, after a 12-week implantation of a hydrogel system containing N-cadherin mimetic peptides in rabbit femoral condyle defects, the hydrogel was associated with significantly higher histological measures of overall defect filling, cartilage surface regularity, GAGs/cell content in newly formed and adjacent cartilage compared to control hydrogel without N-cadherin.

Furthermore, N-cadherin peptide hydrogels suppress canonical Wnt signaling in hMSCs by increasing GSK-3β and GSK-3β-mediated degradation of β-catenin so as to decrease the nuclear translocation and of the associated transcriptional activity of β-catenin/LEF-1/TCF complex, thereby enhancing the chondrogenesis of hMSCs.

### BRIEF SUMMARY

Disclosed are methods of generating successful cartilage grafts that result in better adaptation of artificial cartilage in subjects.

Disclosed are methods of treating a subject having a cartilage graft comprising applying alternating electric fields, at a frequency for a period of time, to a target site of the subject, wherein the target site comprises the cartilage graft.

Disclosed are methods of increasing N-cadherin expression in a subject having a cartilage graft comprising applying alternating electric fields, at a frequency for a period of time, to a target site of the subject, thereby increasing N-cadherin expression at the target site in the subject.

Disclosed are methods of promoting chondrogenesis in a subject having a cartilage graft comprising applying alternating electric fields, at a frequency for a period of time, to a target site of the subject, thereby promoting chondrogenesis in the subject.

Disclosed are methods of upregulating the Akt/PI3K pathway in a subject having a cartilage graft comprising applying alternating electric fields, at a frequency for a period of time, to a target site of the subject.

Additional advantages of the disclosed methods and compositions will be set forth in part in the description which follows, and in part will be understood from the description, or may be learned by practice of the disclosed methods and compositions. The advantages of the disclosed methods and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows that N-cadherin expression is increased in ECM secreting cells (fibroblasts) following TTFields application.

### DETAILED DESCRIPTION

The disclosed methods and compositions may be understood more readily by reference to the following detailed description of particular embodiments and the Example included therein and to the FIGURES and their previous and following description.

It is to be understood that the disclosed methods and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Headings are provided for convenience only and are not to be construed to limit the invention in any manner. Embodiments illustrated under any heading or in any portion of the disclosure may be combined with embodiments illustrated under the same or any other heading or other portion of the disclosure.

### A. Definitions

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a target site” includes a plurality of such target sites, reference to “the cartilage graft” can be a reference to one or more cartilage grafts and equivalents thereof known to those skilled in the art, and so forth.

As used herein, a “target site” is a specific site or location within or present on a subject or patient. For example, a “target site” can refer to, but is not limited to a cartilage graft. For example, the phrase “target cell” can be used to refer to target site, wherein the target site is a cell. In some aspects, a “target cell” can be a chondrogenic cell. In some aspects, sites that can be target sites include, but are not limited to, bone or joints. In some aspects, a cell or population of cells that can be a target site or a target cell include, but are not limited to, a chondrocyte or mesenchymal cell. In some aspects, a target site can refer to a site or location within or present on a subject or patient that comprises a cartilage graft. Additionally, a target site can refer to a site or location adjacent to a cartilage graft.

As used herein, an “alternating electric field” or “alternating electric fields” refers to a very-low-intensity, directional, intermediate-frequency alternating electrical field delivered to a subject, a sample obtained from a subject or to a specific location within a subject or patient (e.g., a target site such as a cartilage graft). In some aspects, the alternating electrical field can be in a single direction or multiple directional. In some aspects, alternating electric fields can be delivered through two pairs of transducer arrays that generate perpendicular fields within the target site. For example, for the Optune® system (an alternating electric fields delivery system) one pair of electrodes is located to the left and right (LR) of the target site, and the other pair of electrodes is located anterior and posterior (AP) to the target site. Cycling the field between these two directions (i.e., LR and AP) ensures that a maximal range of cell orientations is targeted.

In-vivo and in-vitro studies show that the efficacy of alternating electric field therapy increases as the intensity of the electrical field increases. Therefore, optimizing array placement on a subject to increase the intensity in the target site or target cell is standard practice for the Optune system. Array placement optimization may be performed by “rule of thumb” (e.g., placing the arrays on the subject as close to the target site or target cell as possible), measurements describing the geometry of the patient's body, target site dimensions, and/or target site or cell location. Measurements used as input may be derived from imaging data. Imaging data is intended to include any type of visual data, such as for example, single-photon emission computed tomography (SPECT) image data, x-ray computed tomography (x-ray CT) data, magnetic resonance imaging (MRI) data, positron emission tomography (PET) data, data that can be captured by an optical instrument (e.g., a photographic camera, a charge-coupled device (CCD) camera, an infrared camera, etc.), and the like. In certain implementations, image data may include 3D data obtained from or generated by a 3D scanner (e.g., point cloud data). Optimization can rely on an understanding of how the electrical field distributes within the target site or target cell as a function of the positions of the array and, in some aspects, take account for variations in the electrical property distributions within the heads of different patients.

The term “subject” refers to the target of administration, e.g., an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field. For example, the subject of administration can be a subject with a cartilage graft.

By “treat” is meant to administer or apply a therapeutic, such as alternating electric fields and a cartilage graft, to a subject, such as a human or other mammal (for example, an animal model), that has an articular cartilage defect or has an increased susceptibility for developing an articular cartilage defect, in order to prevent or delay a worsening of the effects of the articular cartilage defect, or to partially or fully reverse the effects of an articular cartilage defect. For example, treating a subject having an articular cartilage defect can comprise applying an alternating electric field and/or delivering a therapeutic to a cell or other target site in the subject.

By “prevent” is meant to minimize or decrease the chance that a subject develops a disease or condition, e.g., an articular cartilage defect.

As used herein, the terms “administering” and “administration” refer to any method of providing a therapeutic to a subject directly or indirectly to a target site. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to help with a disease or condition, e.g., grafts. In further various aspects, a preparation can be administered prophylactically; that is, administered for prevention of a disease or condition, e.g., osteoarthritis. In an aspect, the skilled person can determine an efficacious dose, an efficacious schedule, or an efficacious route of administration so as to treat a subject. In some aspects, administering comprises exposing or applying. Thus, in some aspects, exposing a target site or subject to alternating electrical fields or applying alternating electrical fields to a target site or subject means administering alternating electrical fields to the target site or subject.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed methods and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Alternating Electric Fields

The methods disclosed herein comprise alternating electric fields. In some aspects, the alternating electric field used in the methods disclosed herein is a tumor-treating field. In some aspects, the alternating electric field can vary dependent on the type of cell or condition to which the alternating electric field is applied. In some aspects, the alternating electric field can be applied through one or more electrodes placed on the subject's body. In some aspects, there can be two or more pairs of electrodes. For example, arrays can be placed on the front/back and sides of a patient and can be used with the systems and methods disclosed herein. In some aspects, where two pairs of electrodes are used, the alternating electric field can alternate between the pairs of electrodes. For example, a first pair of electrodes can be placed on the front and back of the subject and a second pair of electrodes can be placed on either side of the subject, the alternating electric field can then be applied and can alternate between the front and back electrodes and then to the side to side electrodes.

In some aspects, the frequency of the alternating electric field is between 100 and 500 kHz. In some aspects, the frequency of the alternating electric field is between 50 kHz and 10 MHz. The frequency of the alternating electric fields can also be, but is not limited to, between 50 kHz and 1 MHz, between 50 and 500 kHz, between 100 and 500 kHz, between 25 kHz and 1 MHz, between 50 and 190 kHz, between 25 and 190 kHz, between 180 and 220 kHz, or between 210 and 400 kHz. In some aspects, the frequency of the alternating electric fields can be electric fields at 50 kHz, 100 kHz, 150 kHz, 200 kHz, 250 kHz, 300 kHz, 350 kHz, 400 kHz, 450 kHz, 500 kHz, or any frequency between. In some aspects, the frequency of the alternating electric field is from about 200 kHz to about 400 kHz, from about 250 kHz to about 350 kHz, and may be around 300 kHz.

In some aspects, the field strength of the alternating electric fields can be between 0.5 and 10 V/cm RMS. In some aspects, the field strength of the alternating electric fields can be between 1 and 4 V/cm RMS. In some aspects, different field strengths can be used (e.g., between 0.1 and 10 V/cm). In some aspects, the field strength can be 1.75 V/cm RMS. In some embodiments the field strength is at least 1 V/cm RMS. In some aspects, the field strength can be 0.9 V/cm RMS. In other embodiments, combinations of field strengths are applied, for example combining two or more frequencies at the same time, and/or applying two or more frequencies at different times.

In some aspects, the alternating electric fields can be applied for a variety of different intervals ranging from 0.5 hours to 72 hours. In some aspects, a different duration can be used (e.g., between 0.5 hours and 14 days). In some aspects, application of the alternating electric fields can be repeated periodically. For example, the alternating electric fields can be applied every day for a two hour duration.

In some aspects, the exposure may last for at least 6 hours, at least 12 hours, at least 24 hours, at least 36 hours, at least 48 hours, or at least 72 hours or more.

The disclosed methods comprise applying one or more alternating electric fields to a cell or to a subject. In some aspects, the alternating electric field is applied to a target site. When applying alternating electric fields to a cell, this can often refer to applying alternating electric fields to a subject comprising a cartilage graft. Thus, applying alternating electric fields to a target site of a subject results in applying alternating electric fields to a cell and/or a cartilage graft.

### C. Methods of Treating

Disclosed are methods of treating a subject having a cartilage graft comprising applying alternating electric fields, at a frequency for a period of time, to a target site of the subject, wherein the target site comprises the cartilage graft.

Disclosed are methods of treating a subject requiring a cartilage graft comprising administering a cartilage graft to the subject; and applying alternating electric fields, at a frequency for a period of time, to a target site of the subject, wherein the target site comprises the cartilage graft.

In some aspects, the target site can be a joint such as a knee or shoulder. In some aspects, the target site can be, but is not limited to, ear, nose, knee, shoulder, breast, or hip

In some aspects, the disclosed methods increase the success rate of the graft. In some aspects, the success rate of the graft is determined by allowing a patient to return to pain-free activities. Thus, in some aspects increasing the success rate of the graft means a quicker return to pain-free activities for a subject receiving a cartilage graft. In some aspects, the success rate of the graft is a shortened time to recovery compared to graft alone without alternating electric fields. In some aspects, the success rate of the graft is determined by the number of live cells. In some aspects, the number of live cells in a cartilage graft treated with alternating electric fields can be compared with the number of live cells in a cartilage graft not treated with alternating electric fields.

In some aspects, the alternating electric fields promote chondrogenesis within the cartilage graft. In some aspects, the alternating electric fields increase survival of cells in the cartilage graft.

In some aspects, treating can mean increasing the success rate of a cartilage graft. Thus, also disclosed are methods of increasing the success rate of a cartilage graft in a subject having a cartilage graft comprising applying alternating electric fields, at a frequency for a period of time, to a target site of the subject, wherein the target site comprises the cartilage graft, thereby increasing the success rate of the cartilage graft. Success rate can be determined in one or more of the ways described herein.

Because the target site comprises the cartilage graft, in some aspects, the alternating electric fields are applied directly to the cartilage graft.

In some aspects, the cartilage graft is a scaffold that mimics articular cartilage. In some aspects, a scaffold for cartilage grafts can be made of synthetic or natural polymers or a combination thereof. In some aspects, the cartilage graft is an autologous chondrocyte implantation (ACI). In some aspects, the cartilage graft can be an autograft, allograft, or xenograft. In some aspects, the cartilage graft comprises cells, such as chondrogenic cells and mesenchymal stem cells. In some aspects, a cartilage graft comprises a scaffold, such as a biomaterial scaffold as described herein. In some aspects, a biomaterial scaffold can be biodegradable and/or biocompatible for biomedical application. Furthermore, biomaterial scaffolds can be designed to mimic both compositions and architectures of articular cartilage extracellular matrix (ECM) so as to support the adhesion, migration, proliferation, and differentiation of chondrogenic cells.

In some aspects, the subject receives the cartilage graft at least 24 hours before applying the alternating electric fields. Thus, in some aspects, alternating electric fields are applied to the subject at least 12, 24, 36, 48, 60, or 72 hrs after the subject receives the cartilage graft. In some aspects, the alternating electric fields are applied after the proliferative stage of the cells in the cartilage graft. For example, in some aspects, the alternating electric fields are applied 2, 3, 4, or 5 days after implantation of the cartilage graft.

In some aspects, the methods further comprise administering a therapeutic. For example, in some aspects, the methods further comprise administering an anti-inflammatory, an antibiotic, an osteoarthritis drug, or a pain management medication. In some aspects, the therapeutic can be, but is not limited to, ibuprofen, naproxen, ofloxacin, ciprofloxacin, LNA043.

In some aspects, the subject that receives the cartilage graft does not have cancer and/or has not been diagnosed with cancer. Thus, in some aspects, alternating electric fields are applied to a subject that does not have cancer and/or has not yet been diagnosed with cancer. In some aspects, the target site does not comprise cancer cells and/or a tumor(s).

In some aspects, the methods can further comprise a step of implanting or administering the cartilage graft in the subject prior to applying the alternating electric fields.

In some aspects, the alternating electric fields can have any of the frequencies or field strengths described herein. In some aspects, the frequency of the alternating electric field is between 100 kHz and 10 MHz. For example, the frequency of the alternating electric field may be between 50 kHz and 1 MHz or between 100 kHz and 500 kHz. In some aspects, the frequency of the alternating electric field is 100-300 KHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

In some aspects, the methods can further comprise ceasing application of the alternating electrical field once the cartilage graft is settled. In some aspects, a cartilage graft that is settled means the subject is without pain and/or has normal range of motion. Thus, in some aspects, once the subject is without pain and/or has normal range of motion, the alternating electrical field can be removed or ceased.

Disclosed are compositions comprising a cartilage graft for use in a method of treating a subject, the method comprising introducing the composition to a target site of a subject, and applying an alternating electric field to the target site of the subject.

In some aspects, the composition further comprises mesenchymal stem cells. In some aspects, the cartilage graft comprises mesenchymal stem cells. In some aspects, the method further comprises administering mesenchymal stem cells to the target site of the subject simultaneously with introducing the composition to the target site of a subject.

In some aspects, the alternating electric field promotes chondrogenesis within the cartilage graft, thereby increasing the success rate of the graft. In some aspects, the chondrogenesis is of the MSCs present in a cartilage graft.

In some aspects, the compositions can further comprise an anti-inflammatory, an antibiotic, an osteoarthritis drug, or a pain management medication.

In some aspects, the methods can further comprise administering an anti-inflammatory, an antibiotic, an osteoarthritis drug, or a pain management medication in combination with the composition.

Disclosed are compositions comprising a cartilage graft for use in methods of increasing the success rate of a cartilage graft in a subject having a cartilage graft comprising introducing the composition to a target site of a subject, applying alternating electric fields to the target site of the subject, wherein the target site comprises the cartilage graft, thereby increasing the success rate of the cartilage graft.

### D. Methods of Increasing N-Cadherin

Disclosed herein are methods of increasing N-cadherin in a subject. In some aspects, the increase in N-cadherin is an increase in endogenous N-cadherin in a subject.

Disclosed are methods of increasing N-cadherin expression in a subject having a cartilage graft comprising applying alternating electric fields, at a frequency for a period of time, to a target site of the subject, thereby increasing N-cadherin expression at the target site in the subject.

In some aspects, the target site comprises the cartilage graft. In some aspects, the target site can be a joint such as a knee or shoulder. In some aspects, the target site can be, but is not limited to, ear, nose, knee, shoulder, breast, or hip. Because the target site can comprise the cartilage graft, in some aspects, the alternating electric fields are applied directly to the cartilage graft.

In some aspects, increasing N-cadherin expression at the target site can be any increase compared to no alternating electric fields. In some aspects, increasing N-cadherin expression at the target site can be a 0.5, 1, 1.5, 2, 2.5 or greater increase compared to no alternating electric fields.

In some aspects, increasing N-cadherin in a subject having a cartilage graft increases the success rate of the graft. In some aspects, the success rate of the graft is determined by allowing patients to return to pain-free activities. Thus, in some aspects increasing the success rate of the graft means a quicker return to pain-free activities for a subject receiving a cartilage graft. In some aspects, the success rate of the graft is a shortened time to recovery compared to graft alone without alternating electric fields. In some aspects, the success rate of the graft is determined by the number of live cells. In some aspects, the number of live cells in a cartilage graft treated with alternating electric fields can be compared with the number of live cells in a cartilage graft not treated with alternating electric fields.

In some aspects, the induction of N-cadherin expression by applying alternating electric fields to a subject allows for the removal or reduction of N-cadherin expression by removing (or halting the application of) the alternating electric fields. In some aspects, continuous N-cadherin signaling can be harmful and/or induce cancer formation, therefore the disclosed methods of increasing N-cadherin using alternating electric fields so that removal of the alternating electric fields decreases N-cadherin can be an effective method. Thus, in some aspects, the methods can further comprise ceasing application of the alternating electrical field once the cartilage graft is settled. In some aspects, a cartilage graft that is settled means the subject is without pain and/or has normal range of motion. Thus, in some aspects, once the subject is without pain and/or has normal range of motion, the alternating electrical field can be removed or ceased.

In some aspects, the cartilage graft is a scaffold that mimics articular cartilage. In some aspects, a scaffold for cartilage grafts can be made of synthetic or natural polymers or a combination thereof. In some aspects, the cartilage graft is an autologous chondrocyte implantation. In some aspects, the cartilage graft can be an autograft, allograft, or xenograft. In some aspects, the cartilage graft comprises cells, such as chondrogenic cells and mesenchymal stem cells. In some aspects, a cartilage graft comprises a scaffold, such as a biomaterial scaffold as described herein. In some aspects, a biomaterial scaffold can be biodegradable and/or biocompatible for biomedical application. In some aspects, biomaterial scaffolds can include, but are not limited to, hydrogels, biodegradable scaffolds, micro/nanofibers as instructive biomaterials, and drug-delivering biomaterials. For example, a biomaterial scaffold can be one or more of those scaffolds described in Suzuki et al. Current Concepts of Biomaterial Scaffolds and Regenerative Therapy for Spinal Cord Injury. Int. J. Mol. Sci. 2023, 24 (3), 2528, incorporated by reference in its entirety herein. Furthermore, biomaterial scaffolds can be designed to mimic both compositions and architectures of articular cartilage extracellular matrix so as to support the adhesion, migration, proliferation, and differentiation of chondrogenic cells.

In some aspects, the subject receives the cartilage graft at least 24 hours before applying the alternating electric fields. Thus, in some aspects, alternating electric fields are applied to the subject at least 12, 24, 36, 48, 60, or 72 hrs after the subject receives the cartilage graft. In some aspects, the alternating electric fields are applied after the proliferative stage of the cells in the cartilage graft. For example, in some aspects, the alternating electric fields are applied 2, 3, 4, or 5 days after implantation of the cartilage graft.

In some aspects, the subject that receives the cartilage graft does not have cancer and/or has not been diagnosed with cancer. Thus, in some aspects, alternating electric fields are applied to a subject that does not have cancer and/or has not yet been diagnosed with cancer. In some aspects, the target site does not comprise cancer cells and/or a tumor(s).

In some aspects, the methods can further comprise a step of implanting or administering the cartilage graft in the subject prior to applying the alternating electric fields.

In some aspects, the alternating electric fields can have any of the frequencies or field strengths described herein. In some aspects, the frequency of the alternating electric field is between 100 kHz and 10 MHz. For example, the frequency of the alternating electric field may be between 50 kHz and 1 MHz or between 100 kHz and 500 kHz. In some aspects, the frequency of the alternating electric field is 100-300 kHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

Disclosed are compositions comprising a cartilage graft for use in methods of increasing N-cadherin expression in a subject having a cartilage graft comprising introducing the composition to a target site of a subject, applying alternating electric fields to the target site of the subject, thereby increasing N-cadherin expression at the target site in the subject.

### E. Methods of Promoting Chondrogenesis

Disclosed are methods of promoting chondrogenesis in a subject having a cartilage graft comprising applying alternating electric fields, at a frequency for a period of time, to a target site of the subject, thereby promoting chondrogenesis in the subject.

In some aspects, the target site comprises the cartilage graft. In some aspects, the target site can be a joint such as a knee or shoulder. In some aspects, the target site can be, but is not limited to, ear, nose, knee, shoulder, breast, or hip. Because the target site can comprise the cartilage graft, in some aspects, the alternating electric fields are applied directly to the cartilage graft.

In some aspects, promoting chondrogenesis in a subject having a cartilage graft increases the success rate of the graft. In some aspects, the success rate of the graft is determined by allowing patients to return to pain-free activities. Thus, in some aspects increasing the success rate of the graft means a quicker return to pain-free activities for a subject receiving a cartilage graft. In some aspects, the success rate of the graft is a shortened time to recovery compared to graft alone without alternating electric fields. In some aspects, the success rate of the graft is determined by the number of live cells. In some aspects, the number of live cells in a cartilage graft treated with alternating electric fields can be compared with the number of live cells in a cartilage graft not treated with alternating electric fields.

In some aspects, the cartilage graft is a scaffold that mimics articular cartilage. In some aspects, a scaffold for cartilage grafts can be made of synthetic or natural polymers or a combination thereof. In some aspects, the cartilage graft is an autologous chondrocyte implantation (ACI). In some aspects, the cartilage graft can be an autograft, allograft, or xenograft. In some aspects, the cartilage graft comprises cells, such as chrondrogenic cells and mesenchymal stem cells. In some aspects, a cartilage graft comprises a scaffold, such as a biomaterial scaffold as described herein. In some aspects, a biomaterial scaffold can be biodegradable and/or biocompatible for biomedical application. In some aspects, biomaterial scaffolds can include, but are not limited to, hydrogels, biodegradable scaffolds, micro/nanofibers as instructive biomaterials, and drug-delivering biomaterials. For example, a biomaterial scaffold can be one or more of those scaffolds described in Suzuki et al. Current Concepts of Biomaterial Scaffolds and Regenerative Therapy for Spinal Cord Injury. Int. J. Mol. Sci. 2023, 24 (3), 2528, incorporated by reference in its entirety herein. Furthermore, biomaterial scaffolds can be designed to mimic both compositions and architectures of articular cartilage extracellular matrix (ECM) so as to support the adhesion, migration, proliferation, and differentiation of chondrogenic cells.

In some aspects, the subject receives the cartilage graft at least 24 hours before applying the alternating electric fields. Thus, in some aspects, alternating electric fields are applied to the subject at least 12, 24, 36, 48, 60, or 72 hrs after the subject receives the cartilage graft. In some aspects, the alternating electric fields are applied after the proliferative stage of the cells in the cartilage graft. For example, in some aspects, the alternating electric fields are applied 2, 3, 4, or 5 days after implantation of the cartilage graft.

In some aspects, the subject that receives the cartilage graft does not have cancer and/or has not been diagnosed with cancer. Thus, in some aspects, alternating electric fields are applied to a subject that does not have cancer and/or has not yet been diagnosed with cancer. In some aspects, the target site does not comprise cancer cells and/or a tumor(s).

In some aspects, the methods can further comprise a step of implanting or administering the cartilage graft in the subject prior to applying the alternating electric fields.

In some aspects, the alternating electric fields can have any of the frequencies or field strengths described herein. In some aspects, the frequency of the alternating electric field is between 100 kHz and 10 MHz. For example, the frequency of the alternating electric field may be between 50 kHz and 1 MHz or between 100 kHz and 500 kHz. In some aspects, the frequency of the alternating electric field is 100-300 kHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

Disclosed are compositions comprising a cartilage graft for use in methods of promoting chondrogenesis in the cartilage graft comprising introducing the composition to a target site of a subject, applying alternating electric fields to the target site of the subject, thereby promoting chondrogenesis in the cartilage graft of the subject.

### F. Methods of Upregulating the Akt/PI3K Pathway

Disclosed are methods of upregulating the Akt/PI3K pathway in a subject having a cartilage graft comprising applying alternating electric fields, at a frequency for a period of time, to a target site of the subject.

In some aspects, the disclosed methods of upregulating the Akt/PI3K pathway can increase survival of cells in the cartilage graft.

In some aspects, the target site comprises the cartilage graft. In some aspects, the target site can be a joint such as a knee or shoulder. In some aspects, the target site can be, but is not limited to, ear, nose, knee, shoulder, breast, or hip. Because the target site can comprise the cartilage graft, in some aspects, the alternating electric fields are applied directly to the cartilage graft.

In some aspects, upregulating the Akt/PI3K pathway in a subject having a cartilage graft increases the success rate of the graft. In some aspects, the success rate of the graft is determined by allowing patients to return to pain-free activities. Thus, in some aspects increasing the success rate of the graft means a quicker return to pain-free activities for a subject receiving a cartilage graft. In some aspects, the success rate of the graft is a shortened time to recovery compared to graft alone without alternating electric fields. In some aspects, the success rate of the graft is determined by the number of live cells. In some aspects, the number of live cells in a cartilage graft treated with alternating electric fields can be compared with the number of live cells in a cartilage graft not treated with alternating electric fields.

In some aspects, the cartilage graft is a scaffold that mimics articular cartilage. In some aspects, a scaffold for cartilage grafts can be made of synthetic or natural polymers or a combination thereof. In some aspects, the cartilage graft is an autologous chondrocyte implantation (ACI). In some aspects, the cartilage graft can be an autograft, allograft, or xenograft. In some aspects, the cartilage graft comprises cells, such as chondrogenic cells and mesenchymal stem cells. In some aspects, a cartilage graft comprises a scaffold, such as a biomaterial scaffold as described herein. In some aspects, a biomaterial scaffold can be biodegradable and/or biocompatible for biomedical application. In some aspects, biomaterial scaffolds can include, but are not limited to, hydrogels, biodegradable scaffolds, micro/nanofibers as instructive biomaterials, and drug-delivering biomaterials. For example, a biomaterial scaffold can be one or more of those scaffolds described in Suzuki et al. Current Concepts of Biomaterial Scaffolds and Regenerative Therapy for Spinal Cord Injury. Int. J. Mol. Sci. 2023, 24 (3), 2528, incorporated by reference in its entirety herein. Furthermore, biomaterial scaffolds can be designed to mimic both compositions and architectures of articular cartilage extracellular matrix (ECM) so as to support the adhesion, migration, proliferation, and differentiation of chondrogenic cells.

In some aspects, the subject receives the cartilage graft at least 24 hours before applying the alternating electric fields. Thus, in some aspects, alternating electric fields are applied to the subject at least 12, 24, 36, 48, 60, or 72 hrs after the subject receives the cartilage graft. In some aspects, the alternating electric fields are applied after the proliferative stage of the cells in the cartilage graft. For example, in some aspects, the alternating electric fields are applied 2, 3, 4, or 5 days after implantation of the cartilage graft

In some aspects, the subject that receives the cartilage graft does not have cancer and/or has not been diagnosed with cancer. Thus, in some aspects, alternating electric fields are applied to a subject that does not have cancer and/or has not yet been diagnosed with cancer. In some aspects, the target site does not comprise cancer cells and/or a tumor.

In some aspects, the methods can further comprise a step of implanting or administering the cartilage graft in the subject prior to applying the alternating electric fields.

In some aspects, the alternating electric fields can have any of the frequencies or field strengths described herein. In some aspects, the frequency of the alternating electric field is between 50 kHz and 10 MHz. For example, the frequency of the alternating electric field may be between 50 kHz and 1 MHz or between 100 kHz and 500 kHz. In some aspects, the frequency of the alternating electric field is 100-300 kHz. In some aspects, the frequency of the alternating electric field is 210-400 kHz. In some aspects, the alternating electric field has a field strength of between 0.5 and 10 V/cm RMS. In some aspects, the alternating electric field has a field strength of 0.9 V/cm RMS.

Disclosed are compositions comprising a cartilage graft for use in methods of upregulating the Akt/PI3K pathway in a subject comprising introducing the composition to a target site of a subject, applying alternating electric fields to the target site of the subject, thereby increasing survival of cells in the cartilage graft.

[symbol]

For the new claims include “In some aspects, the methods can further comprise the step of introducing a composition comprising a cartilage graft to a target site of a subject. In some aspects, introducing the composition comprising a cartilage graft to a target site of a subject comprises implanting the cartilage graft to a target site of a subject.”

### G. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising one or more cartilage grafts and one or more materials for delivering alternating electric fields, such as the Optune system.

### Embodiments

Embodiment 1: A method of treating a subject having a cartilage graft comprising applying alternating electric fields, at a frequency for a period of time, to a target site of the subject, wherein the target site comprises the cartilage graft.
Embodiment 2: The method of embodiment 1, wherein the alternating electric fields promote chondrogenesis within the cartilage graft, thereby increasing the success rate of the cartilage graft.
Embodiment 3: A method of increasing N-cadherin expression in a subject having a cartilage graft comprising applying alternating electric fields, at a frequency for a period of time, to a target site of the subject, wherein the target site comprises the cartilage graft, thereby increasing N-cadherin expression at the target site in the subject.
Embodiment 4: A method of promoting chondrogenesis in a subject having a cartilage graft comprising applying alternating electric fields, at a frequency for a period of time, to a target site of the subject, wherein the target site comprises the cartilage graft, thereby promoting chondrogenesis in the subject.
Embodiment 5: A method of increasing the success rate of a cartilage graft in a subject having a cartilage graft comprising applying alternating electric fields, at a frequency for a period of time, to a target site of the subject, wherein the target site comprises the cartilage graft, thereby increasing the success rate of the cartilage graft.
Embodiment 6: A method of upregulating the Akt/PI3K pathway in a subject having a cartilage graft comprising applying alternating electric fields, at a frequency for a period of time, to a target site of the subject, wherein the target site comprises the cartilage graft, thereby increasing survival of cells in the cartilage graft.
Embodiment 7: The method of any of the preceding embodiments, wherein the cartilage graft is a scaffold that mimics articular cartilage or autologous chondrocyte implantation.
Embodiment 8: The method of any of the preceding embodiments, wherein the cartilage graft comprises mesenchymal stem cells.
Embodiment 9: The method of any of the preceding embodiments, wherein the subject received the cartilage graft at least 24 hours before applying the alternating electric fields.
Embodiment 10: The method of any of the preceding embodiments, further comprising administering an anti-inflammatory, an antibiotic, an osteoarthritis drug, or a pain management medication to the subject.
Embodiment 11: The method of any of the preceding embodiments, further comprising implanting the cartilage graft prior to applying the alternating electric fields.
Embodiment 12: The method of any of the preceding embodiments, wherein the subject does not have cancer or has not been diagnosed with cancer.
Embodiment 13: The method of any of the preceding embodiments, wherein the frequency of the alternating electric field is between 100 kHz and 10 MHZ.
Embodiment 14: The method of any of the preceding embodiments, wherein the frequency of the alternating electric field is 210-400 kHz.
Embodiment 15: The method of any of the preceding embodiments, wherein the alternating electric field has a field strength of between 0.5 and 10 V/cm RMS.
Embodiment 16: The method of any of the preceding embodiments, wherein the alternating electric field has a field strength of 0.9 V/cm RMS.
Embodiment 17: The method of any one of embodiments 2-16, further comprising ceasing application of the alternating electrical field once the subject is pain-free and/or has full mobility at the site of the cartilage graft.
Embodiment 18: The method of any of the preceding embodiments, wherein the Akt/PI3K pathway is upregulated.
Embodiment 19: The method of any of the preceding embodiments, wherein the target site is a joint, ear, nose, or breast.
Embodiment 20: The method of any of the preceding embodiments, wherein N-cadherin expression is increased at the target site.
Embodiment 21: The method of any of the preceding embodiments, wherein increasing the success rate of the cartilage graft is a quicker return to pain-free activities for the subject receiving the cartilage graft compared to a subject receiving a cartilage graft without an alternating electric field.
Embodiment 22: The method of embodiment 1, wherein the alternating electric field promotes chondrogenesis at the target site.
Embodiment 23: A composition comprising a cartilage graft for use in a method of treating a subject, the method comprising introducing the composition to a target site of a subject, and applying an alternating electric field to the target site of the subject.
Embodiment 24: The composition of embodiment 23, wherein the composition further comprises mesenchymal stem cells.
Embodiment 25: The composition of embodiment 23, wherein the cartilage graft comprises mesenchymal stem cells.
Embodiment 26: The composition of embodiment 23, wherein the method further comprises administering mesenchymal stem cells to the target site of the subject simultaneously with introducing the composition to the target site of a subject.
Embodiment 27: The composition of embodiment 23, wherein the alternating electric field promotes chondrogenesis within the cartilage graft, thereby increasing the success rate of the graft.
Embodiment 28: A composition comprising a cartilage graft for use in a method of promoting chondrogenesis in a cartilage graft comprising introducing the composition to a target site of a subject, and applying an alternating electric field to the target site comprising the cartilage graft, wherein the cartilage graft comprises mesenchymal stem cells, thereby promoting chondrogenesis of the mesenchymal stem cells in the cartilage graft.
Embodiment 29: The composition of any one of embodiments 23-28, wherein the cartilage graft is a scaffold that mimics articular cartilage or autologous chondrocyte implantation.
Embodiment 30: The composition of any one of embodiments 23-29, wherein the composition is introduced to the target site of the subject at least 24 hours before applying the alternating electric field to the target site.
Embodiment 31: The composition of any one of embodiments 23-30, wherein the method further comprises administering an anti-inflammatory, an antibiotic, an osteoarthritis drug, or a pain management medication to the subject.
Embodiment 32: The composition of any one of embodiments 23-31, wherein the composition is introduced to the target site of the subject prior to applying the alternating electric field.
Embodiment 33: The composition of any one of embodiments 23-32, wherein the subject does not have cancer or has not been diagnosed with cancer.
Embodiment 34: The composition of any one of embodiments 23-32, or the method of claim 3, wherein the frequency of the alternating electric field is between 100 kHz and 10 MHz, optionally 210-400 kHz.
Embodiment 35: The composition of any one of embodiments 23-34, wherein the alternating electric field has a field strength of between 0.5 and 10 V/cm RMS, optionally 0.9 V/cm RMS.
Embodiment 36: The composition of any one of embodiments 23-35, wherein the method further comprises ceasing application of the alternating electrical field once the subject is pain-free and/or has full mobility at the site of the cartilage graft.
Embodiment 37: A kit comprising a cartilage graft and one or more materials for delivering an alternating electric field to a subject.

### EXAMPLES

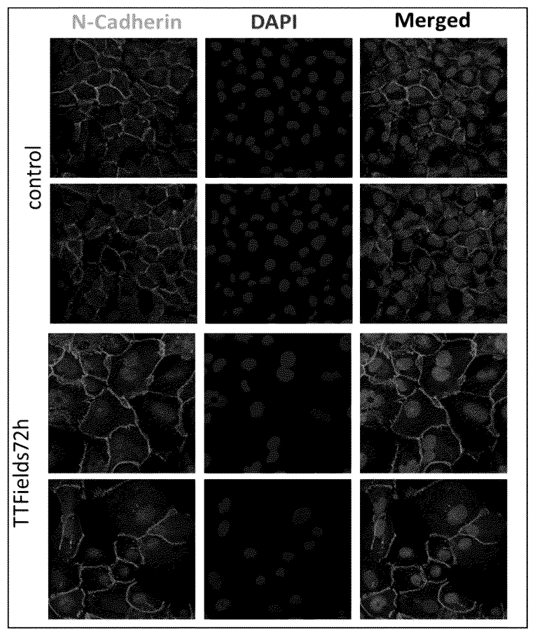

FIG. 1 shows that N-cadherin expression is increased in ECM secreting cells (fibroblasts) following TTFields application.

Biomaterial scaffolds are designed to mimic both compositions and architectures of articular cartilage extracellular matrix (ECM) so as to support the adhesion, migration, proliferation, and differentiation of chondrogenic cells. In some aspects, TTFields can be applied after the proliferative stage of these cells, not directly after implantation of the cells. Thus, TTFields can be applied after a few days since chondrogenic cells have limited cell division.

It was previously known that N-cadherin peptides or mimetics can have a positive effect on cartilage grafts. After a 12-week implantation of a hydrogel system containing N-cadherin mimetic peptides in rabbit femoral condyle defects, the hydrogel was associated with significantly higher histological measures of overall defect filling, cartilage surface regularity, GAGs/cell content in newly formed and adjacent cartilage compared to control hydrogel without N-cadherin.

N-cadherin peptide hydrogels suppress canonical Wnt signaling in hMSCs by increasing GSK-3β and GSK-3β-mediated degradation (we have previously observed GSK3beta activation following TTFields as well) of β-catenin so as to decrease the nuclear translocation and of the associated transcriptional activity of β-catenin/LEF-1/TCF complex, thereby enhancing the chondrogenesis of hMSCs.

Thus, finding a way to increase endogenous N-cadherin, as described herein, is a superior new technology for use in cartilage graft uptake.

An important feature of this invention is the ability to switch on and off the endogenic expression of TTFields, the synthetic N-cadherin scaffold have a limiting half-life that could potentially result in lesser efficacy of graft intake.

Those skilled in the art will recognize, or be able to ascertain using no more than routine experimentation, many equivalents to the specific embodiments of the method and compositions described herein. Such equivalents are intended to be encompassed by the following claims.

We claim:

1. A method of treating a subject having a cartilage graft comprising:
applying alternating electric fields, at a frequency for a period of time, to a target site of the subject,
wherein the target site comprises the cartilage graft.

2. The method of claim 1, wherein the alternating electric fields promote chondrogenesis within the cartilage graft, thereby increasing the success rate of the cartilage graft.

3. A method of increasing N-cadherin expression in a subject having a cartilage graft comprising:
applying alternating electric fields, at a frequency for a period of time, to a target site of the subject, wherein the target site comprises the cartilage graft,
thereby increasing N-cadherin expression at the target site in the subject.

4. A method of promoting chondrogenesis in a subject having a cartilage graft comprising:
applying alternating electric fields, at a frequency for a period of time, to a target site of the subject, wherein the target site comprises the cartilage graft,
thereby promoting chondrogenesis in the subject.

5. The method of claim 1, wherein the cartilage graft is a scaffold that mimics articular cartilage or autologous chondrocyte implantation.

6. The method of claim 1, wherein the cartilage graft comprises mesenchymal stem cells.

7. The method of claim 1, wherein the subject received the cartilage graft at least 24 hours before applying the alternating electric fields.

8. The method of claim 1, further comprising administering an anti-inflammatory, an antibiotic, an osteoarthritis drug, or a pain management medication to the subject.

9. The method of claim 1, further comprising implanting the cartilage graft prior to applying the alternating electric fields.

10. The method of claim 1, wherein the subject does not have cancer or has not been diagnosed with cancer.

11. The method of claim 1, wherein the frequency of the alternating electric field is between 100 kHz and 10 MHz.

12. The method of claim 1, wherein the frequency of the alternating electric field is 210-400 KHz.

13. The method of claim 1, wherein the alternating electric field has a field strength of between 0.5 and 10 V/cm RMS.

14. The method of claim 1, wherein the alternating electric field has a field strength of 0.9 V/cm RMS.

15. The method of claim 2, further comprising ceasing application of the alternating electrical field once the subject is pain-free and/or has full mobility at the site of the cartilage graft.

16. The method of claim 1, wherein the Akt/PI3K pathway is upregulated.

17. The method of claim 1, wherein the target site is a joint, ear, nose, or breast.

18. The method of claim 1, wherein N-cadherin expression is increased at the target site.

19. The method of claim 2, wherein increasing the success rate of the cartilage graft is a quicker return to pain-free activities for the subject receiving the cartilage graft compared to a subject receiving a cartilage graft without an alternating electric field.

20. The method of claim 1, wherein the alternating electric field promotes chondrogenesis at the target site.
